(19) United States
(12) Patent Application Publication
(10) Pub. No.: US 2025/0281138 A1
(43) Pub. Date: Sep. 11, 2025

(54) MEDICAL IMAGE DIAGNOSIS APPARATUS, MEDICAL IMAGE PROCESSING APPARATUS, AND METHOD

(71) Applicant: CANON MEDICAL SYSTEMS CORPORATION, Otawara-shi (JP)

(72) Inventors: Yudai YAMAZAKI, Nasushiobara (JP); Shuki MARUYAMA, Nasushiobara (JP); Yusuke KANO, Edinburgh (GB)

(73) Assignee: CANON MEDICAL SYSTEMS CORPORATION, Otawara-shi (JP)

(21) Appl. No.: 19/071,034

(22) Filed: Mar. 5, 2025

(30) Foreign Application Priority Data
Mar. 7, 2024 (JP) 2024-034900

(51) Int. Cl. A61B 6/00; A61B 6/03; A61B 6/42

(52) U.S. Cl. CPC A61B 6/5217; A61B 6/03; A61B 6/4208; A61B 6/5247

(57) ABSTRACT

According to one embodiment, a medical image diagnosis apparatus includes an acquisition unit and an image capturing condition determination unit. The acquisition unit acquires subject information regarding a subject that includes a first medical image obtained by capturing an image of a diagnosis target region of the subject under a first image capturing condition. The image capturing condition determination unit determines a second image capturing condition to capture a second medical image of the subject, based on the subject information, and a learned model obtained by executing learning while associating evaluation information regarding a medical image and an image capturing condition.

### CROSS-REFERENCE TO RELATED APPLICATIONS

This application is based upon and claims the benefit of priority from Japanese Patent Application No. 2024-034900, filed Mar. 7, 2024, the entire contents of which are incorporated herein by reference.

### FIELD

Embodiments described herein relate generally to a medical image diagnosis apparatus, a medical image processing apparatus, and a method.

### BACKGROUND

In conventional image diagnosis examinations, a user, such as a technologist, has determined an image capturing condition, such as an image capturing range, based on an examination order. For example, capturing an image of a whole body of a subject at once regardless of a diagnosis target region allows a sufficient examination to be used for diagnosis, but setting a wide image capturing range is undesirable in view of the case of an examination involving invasive procedures due to radiation exposure and the time cost. For this reason, in conventional image diagnosis examinations, the user has determined an appropriate image capturing range. Because an image capturing condition appropriate for diagnosis depends also on the state of the subject at the time of image capturing, it has been demanded to enable an image capturing condition to be dynamically determined at the time of image capturing.

For example, in a blood examination, there has been known a technique of a model that dynamically determines the type of blood examination to be executed next in such a manner that disease classification accuracy improves, based on a result of the previous blood examination. In an image diagnosis examination, because an image capturing position suitable for a subject is factored in, the method of simply determining the type and the order of examination as in a blood examination has been inapplicable to automatic determination of an image capturing condition.

### BRIEF DESCRIPTION OF THE DRAWINGS

FIG. 1 is a diagram illustrating an example of a configuration of an X-ray computed tomography scanner of one embodiment;

FIG. 2 is a diagram illustrating an example of subject information of the one embodiment;

FIG. 3 is a diagram illustrating an example of evaluation information of the one embodiment;

FIG. 4 is a diagram illustrating an example of an image capturing position in an image diagnosis examination including a plurality of times of image capturing of the one embodiment;

FIG. 5 is a diagram for an explanation of Formula (2) of the one embodiment;

FIG. 6 is a flowchart illustrating an example of a procedure of learning processing of the one embodiment;

FIG. 7 is a flowchart illustrating an example of a procedure of image capturing processing of the one embodiment;

FIG. 8 is a diagram illustrating an example of a configuration of a magnetic resonance imaging apparatus of another embodiment;

FIG. 9 is a flowchart illustrating an example of a procedure of image capturing processing of the another embodiment;

FIG. 10 is a diagram illustrating an example of a configuration of an ultrasound diagnosis apparatus of yet another embodiment; and

FIG. 11 is a diagram illustrating an example of a configuration of a workstation of yet another embodiment.

### DETAILED DESCRIPTION

A medical image diagnosis apparatus of one embodiment includes an acquisition unit and an image capturing condition determination unit. The acquisition unit acquires subject information regarding a subject including a first medical image obtained by capturing an image of a diagnosis target region of the subject under a first image capturing condition. The image capturing condition determination unit determines a second image capturing condition to capture a second medical image of the subject, based on a learned model that has learned the subject information, an evaluation information regarding a medical image, and an image capturing condition in association.

Various embodiments will be described hereinafter with reference to the accompanying drawings.

### One Embodiment

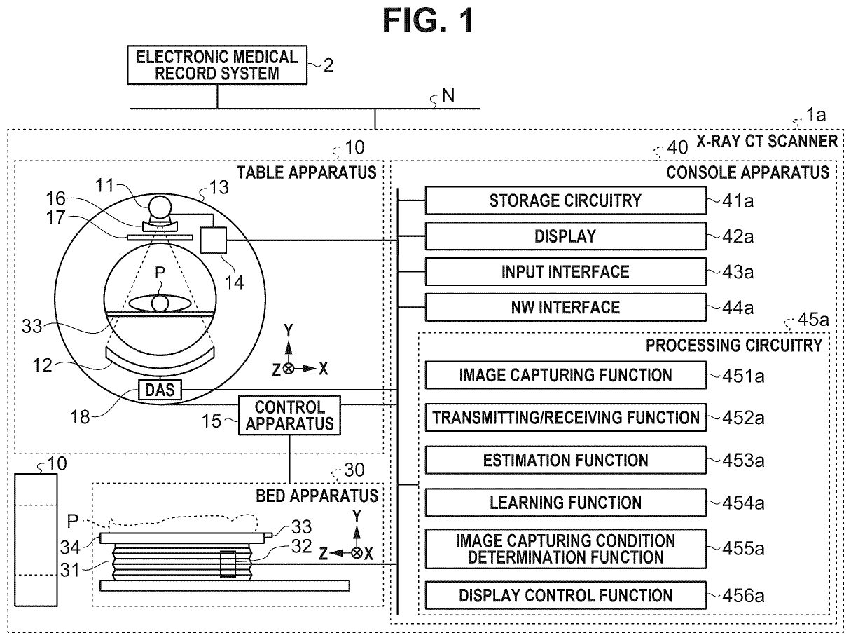

FIG. 1 is a diagram illustrating an example of a configuration of a computed tomography (CT) scanner 1 (hereinafter, will be referred to as an “X-ray CT scanner 1a”) according to one embodiment. The X-ray CT scanner 1a is an example of a medical image diagnosis apparatus according to the one embodiment. The X-ray CT scanner 1a may be referred to as a radiation image diagnosis apparatus. The X-ray CT scanner 1a is installed in a hospital or the like, for example. The configuration of the X-ray CT scanner 1a illustrated in FIG. 1 is an example, and the configuration is not limited to this. For example, the X-ray CT scanner 1a may be configured to capture an image of a subject in an upright position or in a seated position.

During an image diagnosis examination in which image capturing is consecutively executed a plurality of times, the X-ray CT scanner 1a according to the one embodiment automatically determines an appropriate image capturing condition for the next image capturing in accordance with a result of the previous image capturing. The X-ray CT scanner 1a is configured to execute image capturing consecutively at least twice or more. In the one embodiment, in a case where wordings “previous image capturing” and “next image capturing” are used, these wordings respectively mean “previous image capturing” and “next image capturing” within a plurality of times of image capturing in one image diagnosis examination.

As illustrated in FIG. 1, the X-ray CT scanner 1a may connect with an electronic medical record system 2 via a network N, such as an in-hospital local area network (LAN), for example, in such a manner that communication is performed with each other. The X-ray CT scanner 1a may connect with a hospital information system (HIS), a laboratory information system (LIS), a radiology information system (RIS), and a medical image storage apparatus in such a manner that communication is performed with each other.

As illustrated in FIG. 1, the X-ray CT scanner 1a includes a table apparatus 10, a bed apparatus 30, and a console apparatus 40. A subject P (for example, human body) is not included in the X-ray CT scanner 1a.

In the one embodiment, a rotation axis of a rotatable frame 13 in a non-tilt state or a longer direction of a top panel 33 of the bed apparatus 30 is defined as a Z-axis direction, an axis direction that is orthogonal to the Z-axis direction and horizontal to a floor surface, is defined as an X-axis direction, and an axis direction that is orthogonal to the Z-axis direction and vertical to the floor surface is defined as a Y-axis direction. In FIG. 1, for the sake of explanatory convenience, a plurality of table apparatuses 10 are drawn, but as an actual configuration of the X-ray CT scanner 1a, the number of table apparatuses 10 is one.

The table apparatus 10 and the bed apparatus 30 operate based on an operation performed by a user via the console apparatus 40, or an operation performed by the user via an operation unit disposed on the table apparatus 10 or the bed apparatus 30. The table apparatus 10, the bed apparatus 30, and the console apparatus 40 are connected in a wired or wireless manner in such a manner that communication is performed with each other.

The table apparatus 10 is an apparatus including an image capturing system that emits X-rays to the subject P and collects detection data of X-rays transmitted through the subject P. More specifically, the table apparatus 10 includes an X-ray tube 11 (X-ray generation unit), a wedge 16, a collimator 17, an X-ray detector 12, an X-ray high-voltage apparatus 14, a data acquisition system DAS (DAS) 18, the rotatable frame 13, and a control apparatus 15.

The X-ray tube 11 is a vacuum tube that receives application of high voltage and supply of filament current from the X-ray high-voltage apparatus 14 and generates X-rays by emitting thermal electrons from a cathode (filament) toward an anode (target). The collision of thermal electrons with the target generates X-rays. X-rays generated at a tube focal spot of the X-ray tube 11 are formed into a cone-beam shape via the collimator 17, for example, and emitted to the subject P. Examples of the X-ray tube 11 include a rotating anode X-ray tube that generates X-rays by emitting thermal electrons to a rotating anode.

As illustrated in FIG. 1, the X-rays formed into a cone-beam shape have a shape spreading in a fan shape in the X-axis direction. Thus, an angle indicating the spreading in the X-axis direction of X-rays formed into a cone-beam shape will be referred to as a fan angle. An angle indicating the depth in the Z-axis direction of X-rays formed into a cone-beam shape will be referred to as a cone angle. Thus, the X-axis direction will also be referred to as a fan angle direction and the Z-axis direction will also be referred to as a cone angle direction.

The X-ray detector 12 detects X-rays that have been emitted from the X-ray tube 11 and transmitted through the subject P, and outputs an electric signal corresponding to an amount of the X-rays, to the DAS 18.

The X-ray detector 12 includes a plurality of detection element columns on which a plurality of detection elements are arrayed in a channel direction along one arc centered on the tube focal spot of the X-ray tube 11, for example. Each of the plurality of detection elements detects an incident amount of X-rays. Types of the X-ray CT scanner 1a include various types, such as a rotate/rotate-type (third-generation CT) that the X-ray tube 11 and the X-ray detector 12 integrally rotate around the subject P, and a stationary/rotate-type (fourth-generation CT) in which a number of X-ray detection elements arrayed in a ring shape are fixed and only the X-ray tube 11 rotates around the subject P. The X-ray CT scanner 1a of any type is applicable to the one embodiment.

More specifically, the X-ray detector 12 is a direct conversion type X-ray detector including a semiconductor device that converts incident X-rays into electric charges. The X-ray detector 12 according to the one embodiment includes at least one high voltage electrode, at least one semiconductor device, and a plurality of readout electrodes. The semiconductor device will also be referred to as an X-ray conversion device.

The X-ray detector 12 according to the one embodiment may employ whichever of an energy integrated collection method and a photon counting method.

The rotatable frame 13 supports the X-ray tube 11 and the X-ray detector 12 in such a manner that the X-ray tube 11 and the X-ray detector 12 are rotatable around the rotation axis. Specifically, the rotatable frame 13 is an annular frame that supports the X-ray tube 11 and the X-ray detector 12 in positions facing each other, and rotates the X-ray tube 11 and the X-ray detector 12 by the control apparatus 15 to be described below. The rotatable frame 13 is rotatably supported by a fixed frame formed of metal, such as aluminum. In response to receipt of power from a drive mechanism of the control apparatus 15, the rotatable frame 13 rotates around the rotation axis at a fixed angular speed.

In addition to the X-ray tube 11 and the X-ray detector 12, the rotatable frame 13 further supports the X-ray high-voltage apparatus 14 and the DAS 18. The rotatable frame 13 as described above is accommodated in an approximately-cylindrical shape casing having a bore forming an image capturing space. The central axis of the bore coincides with the rotation axis of the rotatable frame 13.

The X-ray high-voltage apparatus 14 includes an electric circuitry, such as a transformer and a rectifier, and includes a high voltage generation apparatus having a function of generating high voltage to be applied to the X-ray tube 11 and filament current to be supplied to the X-ray tube 11, and an X-ray control apparatus that controls output voltage in accordance with X-rays to be emitted by the X-ray tube 11. The high voltage generation apparatus may employ whichever of a converter system and an inverter system. The X-ray high-voltage apparatus 14 may be disposed on the rotatable frame 13, or may be disposed on a fixed frame (not illustrated) of the table apparatus 10. The fixed frame is a frame that rotatably supports the rotatable frame 13.

The control apparatus 15 includes a processing circuitry including a central processing unit (CPU), and a drive mechanism, such as a motor and an actuator. The processing circuitry includes, as hardware resources, a processor, such as a CPU and a micro processing unit (MPU), and a memory, such as a read only memory (ROM) and a random access memory (RAM). The control apparatus 15 may be implemented by a processor, such as a graphics processing unit (GPU), an application specific integrated circuit (ASIC), a programmable logic device (for example, simple programmable logic device (SPLD)), a complex programmable logic device (CPLD), and a field programmable gate array (FPGA). In a case where a processor is a CPU, for example, the processor implements a function by reading out a program stored in a memory, and executing the program. On the other hand, in a case where a processor is an ASIC, instead of storing a program into a memory, the function is directly embedded into a circuitry of the processor as a logic circuitry. The processors according to the one embodiment are not limited to processors each being formed as a single circuitry, and one processor may be formed by combining a plurality of independent circuitries, and its function may be implemented. Furthermore, a plurality of components may be integrated into one processor, and its function may be implemented.

The control apparatus 15 has a function of performing operation control of the table apparatus 10 and the bed apparatus 30 in response to receipt of an input signal from an input interface 43a disposed on the console apparatus 40 or the table apparatus 10. For example, in response to receipt of an input signal, the control apparatus 15 performs control of rotating the rotatable frame 13, control of tilting the table apparatus 10, and control of operating the bed apparatus 30 and the top panel 33. The control of tilting the table apparatus 10 may be implemented by the control apparatus 15 rotating the rotatable frame 13 about an axis parallel to the X-axis direction, based on inclination angle (tilt angle) information input by the input interface 43a disposed on the table apparatus 10. The control apparatus 15 may be disposed on the table apparatus 10 or may be disposed on the console apparatus 40.

The wedge 16 is a filter for adjusting an X-ray amount of X-rays emitted from the X-ray tube 11. For example, the wedge 16 is a wedge filter or a bow-tie filter, or an aluminum filter having a predetermined target angle or a predetermined thickness.

The collimator 17 is, for example, a lead plate for narrowing down X-rays transmitted through the wedge 16, into an X-ray irradiation range, and a slit is formed by the combination of a plurality of lead plates, for example.

The DAS 18 includes an amplifier that performs amplification processing on an electric signal output from each X-ray detection element of the X-ray detector 12, and an analog-to-digital (A/D) converter that converts the electric signal into a digital signal, and the DAS 18 generates detection data. The detection data generated by the DAS 18 is transferred to the console apparatus 40.

In the one embodiment, in a case where a phrase “detection data” is used, the phrase encompasses both meanings of pure raw data that has been detected by the X-ray detector 12 and is not being subjected to preprocessing, and raw data obtained by performing preprocessing on pure raw data. Data (detection data) not being subjected to preprocessing and data having been subjected to preprocessing will be sometimes collectively referred to as projection data.

The bed apparatus 30 is an apparatus for placing or moving the subject P to be scanned, and includes a base 31, a bed driving apparatus 32, the top panel 33, and a top panel support frame 34. The base 31 is a casing that supports the top panel support frame 34 in such a manner that the top panel support frame 34 is movable in a vertical direction. The bed driving apparatus 32 is a motor or an actuator that moves the top panel 33 on which the subject P is placed, in a long axis direction of the top panel 33. The bed driving apparatus 32 moves the top panel 33 in accordance with control performed by the console apparatus 40, or performed by the control apparatus 15. The top panel 33 disposed on the top surface of the top panel support frame 34 is a panel on which the subject P is placed. In addition to the top panel 33, the bed driving apparatus 32 may move the top panel support frame 34 in the long axis direction of the top panel 33.

The console apparatus 40 is an apparatus that executes control of the table apparatus 10, and generation of a CT image that is based on a scan result obtained by the table apparatus 10. The console apparatus 40 includes a storage circuitry 41a (storage unit), a display 42a (display unit), the input interface 43a (input unit), a network (NW) interface 44a, and a processing circuitry 45a (processing unit). Data communication between the storage circuitry 41a, the display 42a, the input interface 43a, the processing circuitry 45a is performed via a bus.

For example, the storage circuitry 41a is implemented by a semiconductor memory device, such as a RAM or a flash memory, a hard disk drive (HDD), a solid state drive (SSD), or an optical disk. The storage circuitry 41a may be a driving apparatus that reads and writes various types of information between a portable storage medium, such as a compact disc (CD), a digital versatile disc (DVD), or a flash memory, and a semiconductor memory device, such as a RAM. The storage circuitry 41a stores, for example, projection data and reconstructed image data. A storage region of the storage circuitry 41a may be provided in the X-ray CT scanner 1a, or may be provided in an external storage apparatus connected via a network. The storage circuitry 41a stores a control program according to the one embodiment. The storage circuitry 41a is an example of a storage unit.

The display 42a displays various types of information. For example, the display 42a outputs a medical image (CT image) generated by the processing circuitry 45a, and a graphical user interface (GUI) for receiving various operations from the user. As the display 42a, for example, a liquid crystal display (LCD), an organic electro luminescence (EL) display (OELD), a plasma display, or any other displays is used as appropriate. The display 42a may be disposed on the table apparatus 10. The display 42a may be of a desktop type, or may be formed by a tablet terminal that wirelessly communicates with the main body of the console apparatus 40.

The input interface 43a receives various input operations from the user, converts the received input operations into electric signals, and outputs the electric signals to the processing circuitry 45a. From the user, the input interface 43a receives, for example, a collection condition to be used in collecting projection data, a reconstruction condition to be used in reconstructing a CT image, and an image processing condition to be used in generating a postprocessing image from the CT image. As the input interface 43a, for example, a mouse, a keyboard, a trackball, a switch, a button, a joystick, a touchpad, and a touch panel display are used as appropriate.

In the one embodiment, the input interface 43a is not limited to an interface that includes a physical operational component, such as a mouse, a keyboard, a trackball, a switch, a button, a joystick, a touchpad, and a touch panel display. For example, a processing circuitry of an electric signal that receives an electric signal corresponding to an input operation, from an external input device disposed separately from an apparatus, and outputs this electric signal to the processing circuitry 45a is also included in the examples of the input interface 43a. The input interface 43a is an example of an input unit. The input interface 43a may be disposed on the table apparatus 10. The input interface 43a may be formed by a tablet terminal that wirelessly communicates with the many body of the console apparatus 40.

The NW interface 44a acquires subject information regarding the subject P from the electronic medical record system 2 or a different apparatus via the network N.

The processing circuitry 45a controls entire operation of the X-ray CT scanner 1a in accordance with an electric signal of an input operation output from the input interface 43a. For example, the processing circuitry 45a includes an image capturing function 451a, a transmitting/receiving function 452a, an estimation function 453a, a learning function 454a, an image capturing condition determination function 455a, and a display control function 456a. Here, for example, processing functions that are executed by the image capturing function 451a, the transmitting/receiving function 452a, the estimation function 453a, the learning function 454a, the image capturing condition determination function 455a, and the display control function 456a, which are components of the processing circuitry 45a illustrated in FIG. 1, are recorded in the storage circuitry 41a in the form of computer-executable programs. The processing circuitry 45a is a processor, for example, and reads out each program from the storage circuitry 41a and implements a function corresponding to the program, by executing the program. In other words, the processing circuitry 45a in a state in which each program is read out has a corresponding function illustrated in the processing circuitry 45a in FIG. 1. The image capturing function 451a is an example of an imaging unit. The transmitting/receiving function 452a is an example of a transmitting/receiving unit and an output unit. The image capturing function 451a and the transmitting/receiving function 452a serve as an example of an acquisition unit. The estimation function 453a is an example of an estimation unit. The learning function 454a is an example of a learning unit. The image capturing condition determination function 455a is an example of an image capturing condition determination unit. The display control function 456a is an example of a display control unit and an output unit.

While FIG. 1 illustrates a case where the image capturing function 451a, the transmitting/receiving function 452a, the estimation function 453a, the learning function 454a, the image capturing condition determination function 455a, and the display control function 456a are implemented by the single processing circuitry, i.e., the processing circuitry 45a, the one embodiment is not limited to this. For example, the processing circuitry 45a may be a combination of a plurality of independent processors, and each processor may implement the corresponding processing function by executing the corresponding program. Each processing function included in the processing circuitry 45a may be implemented by being appropriately allocated to a plurality of processing circuitries or integrated into a single processing circuitry.

The image capturing function 451a executes image capturing of the subject P by controlling various components of the X-ray CT scanner 1a. The image capturing function 451a obtains CT image data as an image capturing result. A CT image indicated by the CT image data serves as an example of a first medical image, a second medical image, and a medical image for learning in the one embodiment. In the one embodiment, in a case where a word “CT image” is used, the CT image is not limited to tomographic image data taken along a certain cross-section, and the word may also refer to a three-dimensional image data.

For example, the image capturing function 451a executes preprocessing, reconstruction, and image processing. Specifically, the image capturing function 451a generates projection data obtained by performing preprocessing, such as logarithmic conversion processing, offset correction processing, inter-channel sensitivity correction processing, and beam hardening correction, on detection data output from the DAS 18 as preprocessing. The image capturing function 451a generates a CT image by performing reconstruction processing that uses a filter correction back projection method or a successive approximation reconstruction method, on the projection data generated by preprocessing. The image capturing function 451a may use a known method to perform various types of image processing on the CT image generated by the reconstruction processing.

An image capturing condition for capturing an image of the subject P is defined by the image capturing condition determination function 455a to be described below. The image capturing function 451a acquires a CT image by capturing an image of the subject P under an image capturing condition defined by the image capturing condition determination function 455a. In first-time image capturing, image capturing may be performed based on a predetermined image capturing condition specified by the user, instead of an image capturing condition defined by the image capturing condition determination function 455a.

The image capturing condition of CT images may include, for example, an image capturing position to which X-rays are emitted, an X-ray dosage, an image parameter, operations of the table apparatus 10 and the bed driving apparatus 32, and an X-ray radiation timing. In the one embodiment, an image capturing condition includes at least the image capturing position.

The transmitting/receiving function 452a may acquire subject information regarding the subject P that excludes CT images, from the electronic medical record system 2 or another apparatus via the network N and the NW interface 44a.

The subject information is information regarding the body of the subject P. More specifically, in the one embodiment, the subject information includes at least a CT image captured by the X-ray CT scanner 1a. Aside from CT image data, the subject information may include, for example, an examination order for the subject P and medical care information recorded on an electronic medical record.

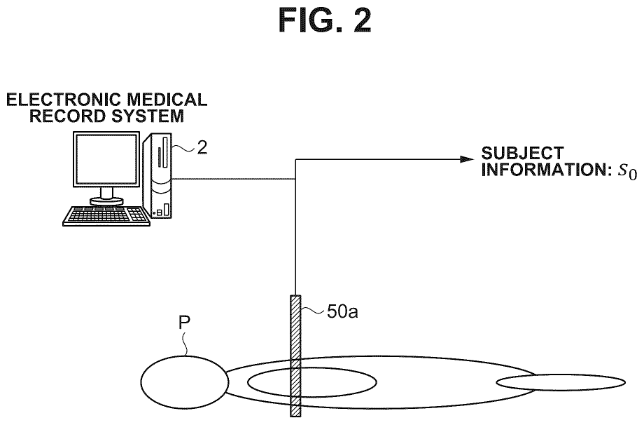

FIG. 2 is a diagram illustrating an example of subject information according to the one embodiment. As illustrated in FIG. 2, the subject information according to the one embodiment includes a CT image obtained by capturing an image of the subject P, and medical care information of the subject P that is acquired from the electronic medical record system 2. The medical care information regarding the subject P includes, for example, a disease name.

In the one embodiment, subject information regarding the subject P at a time t is denoted by Sₜ. In an image diagnosis examination including a plurality of times of image capturing, subject information including a CT image captured in first-time image capturing is denoted by S₀. An image capturing position 50a illustrated in FIG. 2 is an image capturing position in the first-time image capturing of an image diagnosis examination including a plurality of times of image capturing to be executed by the X-ray CT scanner 1a according to the one embodiment. That is to say, the subject information S₀ illustrated in FIG. 2 is subject information at a time point at which the first-time image capturing is completed.

In an image diagnosis examination including a plurality of times of image capturing, each time when the first-time image capturing is completed by the image capturing function 451a, a captured CT image is added to the subject information. For example, after completion of second-time image capturing, a CT image obtained by capturing an image of the subject P in the second-time image capturing is included in the subject information. After completion of third-time image capturing, a CT image obtained by capturing an image of the subject P in the third-time image capturing is included in the subject information. In other words, the subject information according to the one embodiment includes captured CT images of the first-time image capturing to the previous image capturing in an image diagnosis examination including a plurality of times of image capturing.

Referring back to FIG. 1, based on the subject information, the estimation function 453a obtains evaluation information regarding an estimated image estimated to be obtained by the image capturing of a diagnosis target region of the subject P under an image capturing condition different from an image capturing condition used when a CT image included in the subject information has been captured. The different image capturing condition is an image capturing condition different in image capturing position, for example. Because image capturing of the same position is not basically performed a plurality of times in X-ray CT image capturing, the estimation function 453a obtains evaluation information regarding an estimated image estimated to be obtained by the image capturing of a diagnosis target region of the subject P under an image capturing condition different from an image capturing condition used when the CT image included in the subject information has been captured.

More specifically, the estimation function 453a estimates an estimated image based on the subject information. Then, the estimation function 453a generates evaluation information based on the CT image included in the subject information and the estimated image.

The evaluation information according to the one embodiment includes, for example, evaluation information regarding the image quality of the estimated image, and evaluation information regarding uncertainty of disease classification estimated to be obtained in a case where a disease is classified based on the CT image included in the subject information and the estimated image. The evaluation information regarding the image quality of the estimated image is an example of first evaluation information according to the one embodiment. The evaluation information regarding uncertainty of disease classification estimated to be obtained in a case where a disease is classified based on the CT image included in the subject information and the estimated image is an example of second evaluation information according to the one embodiment.

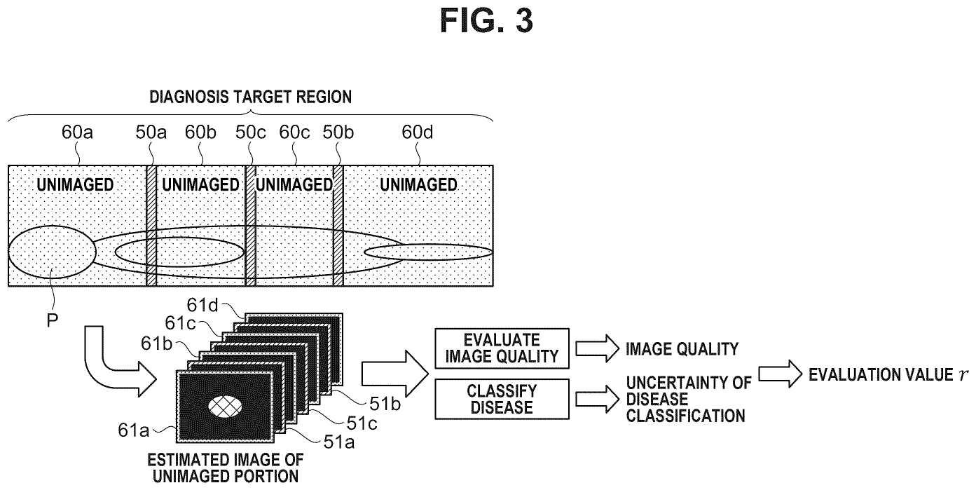

FIG. 3 is a diagram illustrating an example of evaluation information according to the one embodiment. As illustrated in FIG. 3, in the one embodiment, a diagnosis target region of the subject P is a whole body. In the example illustrated in FIG. 3, image capturing has been already executed three times in an image diagnosis examination including a plurality of times of image capturing. Specifically, in the whole body of the subject P that serves as a diagnosis target region, images of three points corresponding to image capturing positions 50a to 50c (hereinafter, in a case where no particular distinction is made among the image capturing positions 50a to 50c, these will be simply referred to as the image capturing positions 50) have already been captured. The estimation function 453a estimates an estimated image estimated to be obtained by image capturing of an image capturing position different from the image capturing positions in the past three times of image capturing.

Specifically, the estimation function 453a estimates estimated images 61a to 61d (hereinafter, in a case where no particular distinction is made among the estimated images 61a to 61d, these will be simply referred to as the estimated images 61) estimated to be obtained by image capturing of unimaged portions 60a to 60d (hereinafter, in a case where no particular distinction is made among the unimaged portions 60a to 60d, these will be simply referred to as the unimaged portions 60). The estimated image 61a is an image estimated to be obtained by X-ray CT scanning of the unimaged portion 60a by the X-ray CT scanner 1a. The estimated image 61b is an image estimated to be obtained by X-ray CT scanning of the unimaged portion 60b by the X-ray CT scanner 1a. The estimated image 61c is an image estimated to be obtained by X-ray CT scanning of the unimaged portion 60c by the X-ray CT scanner 1a. The estimated image 61d is an image estimated to be obtained by X-ray CT scanning of the unimaged portion 60d by the X-ray CT scanner 1a. More specifically, the estimation function 453a estimates the state of the entire diagnosis target region of the subject P based on captured CT images 51, and the estimated images 61 for the unimaged portions 60.

The number of estimated images 61 to be generated for one unimaged portion 60 is not limited to one, and a plurality of estimated images 61 may be generated for one unimaged portion 60. In addition to the image capturing positions, other imaging conditions of the estimated images 61 may be different from the image capturing condition used in the image capturing of the image capturing positions 50a to 50c.

In a case where a diagnosis target region specified in an examination order is not the whole body of the subject P but a part of the body of the subject P, such as an abdominal region or a head region, for example, or a specific organ like heart, the estimation function 453a generates the estimated image 61 for an unimaged portion within the diagnosis target region. For an unimaged portion falling outside the diagnosis target region, the estimation function 453a does not generate the estimated image 61.

A CT image 51a illustrated in FIG. 3 is obtained by image capturing of the image capturing position 50a. A CT image 51b is obtained by image capturing of an image capturing position 50b. A CT image 51c is obtained by image capturing of an image capturing position 50c.

The estimation function 453a evaluates the image quality of the CT images 51a to 51c (hereinafter, in a case where no particular distinction is made among the CT images 51a to 51c, these will be simply referred to as the CT images 51) obtained by the image capturing of the image capturing positions 50a to 50c and the estimated images 61a to 61c. While, in the one embodiment, as an example, the estimation function 453a targets both the CT images 51 and the estimated images 61 in image quality evaluation, the estimation function 453a evaluates the image quality of at least the estimated images 61.

As evaluation information regarding the image quality of the estimated images 61, for example, image quality evaluation values, such as a signal to noise ratio (SNR), a peak signal to noise ratio (PSNR), or structural similarity (SSIM), are usable. The estimation function 453a calculates the SNR, the PSNR, or the SSIM for each of the CT images 51a to 51c and the estimated images 61a to 61c, and calculates an average value of calculation results. The average value serves as an evaluation value of the image quality of estimated images. For example, the image quality of estimated images varies in accordance with an X-ray dosage included in an image capturing condition. The estimation function 453a may employ any one of the SNR, the PSNR, and the SSIM, or may calculate an evaluation value by combining some of the SNR, the PSNR, and the SSIM. The estimation function 453a performs the calculation in such a manner that the evaluation value increases with an increase in the image quality of the estimated images 61.

As the evaluation information regarding uncertainty of disease classification estimated to be obtained in a case where a disease is classified based on the CT image included in the subject information and the estimated image (hereinafter, evaluation information regarding uncertainty of disease classification), the uncertainty of a model in a disease classification model and a report generation model is usable, for example. It can be said that the insufficiency of the information is increased (additional image capturing is required, and/or image capturing under a different condition is required) with an increase in the uncertainty. As disease classification, for example, computer aided detection (CADe), or computer aided diagnosis (CADx) may be employed. If a neural network of the disease classification model or the report generation model is available, uncertainty may be obtained by a Monte Carlo Dropout method. Alternatively, the estimation function 453a may evaluate the uncertainty of disease classification by a rule-based automatic diagnosis method. The estimation function 453a performs the calculation in such a manner that the evaluation value increases with a decrease in the uncertainty of disease classification.

The estimation function 453a calculates an evaluation value r based on evaluation information regarding the image quality of the estimated images 61 and evaluation information regarding uncertainty of disease classification. The evaluation value r increases with an increase in the image quality of the estimated images 61 and decreases with a decrease in the uncertainty of disease classification, for example. Weights of image quality and the uncertainty of disease classification may be set as appropriate. The evaluation value r is an example of the evaluation information according to the one embodiment. While, in the one embodiment, the evaluation value r is calculated as evaluation information, evaluation information may be represented by any form other than a numerical value and may be represented by a rank level.

The estimation function 453a may use a plurality of evaluation methods in combination in image quality evaluation and disease classification evaluation. In addition to the evaluation information regarding the image quality of the estimated images 61 and the evaluation information regarding the uncertainty of disease classification, the estimation function 453a may further use an examination time, a dosage, and a bed moving distance in evaluation. For example, because a time frame of an image diagnosis examination of the subject P is predetermined, the estimation function 453a may use a penalty of decreasing the evaluation value r in a case where an examination time exceeds a predetermined percentage of the time frame by an estimated image capturing condition. Because a dosage of X-rays that is allowed to be emitted to the subject P has an upper limit, the estimation function 453a may use a penalty of decreasing the evaluation value r in a case where an X-ray dosage exceeds a threshold value. As for the threshold value of the X-ray dosage, the estimation function 453a may execute evaluation by factoring in an X-ray dosage in a different examination executed within a predetermined period, not only in an image diagnosis examination of this time. If a bed moving distance to the next image capturing position 50 is long, it takes time in moving, and the length of an examination time is consequently affected in some cases. Thus, the estimation function 453a may use a penalty of decreasing the evaluation value r with an increase in a moving distance from a bed position of an image capturing condition in the previous image capturing.

Referring back to FIG. 1, the learning function 454a generates a learned model by causing a model to execute learning while associating evaluation information regarding a medical image (for example, CT image) and an image capturing condition, in such a manner that the evaluation value r is increased. For example, the learning function 454a causes a model to execute learning while associating a CT image, an estimated image generated from the CT image, the evaluation value r calculated based on the CT image and the estimated image, and an image capturing condition under which the CT image has been captured. More specifically, the learning function 454a causes a model to execute learning in such a manner that the image quality of a medical image improves, and the uncertainty of disease classification estimated to be obtained in a case where a disease is classified based on the medical image is decreased. As the model, for example, a deep learning model, such as a neural network, is usable.

As a method of causing the model to execute learning, reinforcement learning or a machine learning method is usable. As an example of the reinforcement learning, an algorithm of measure-based reinforcement learning may be used. The measure-based reinforcement learning is an algorithm of improving a measure x to obtain a measure that maximizes a value. In the one embodiment, the measure x corresponds to an image capturing condition. In the one embodiment, the learned model may be referred to as an image capturing measure model.

As an example of the measure-based reinforcement learning, the learning function 454a according to the one embodiment may use an Actor-Critic method. The following Formula (1) is an example of a formula representing an algorithm in a case where the learning function 454a performs reinforcement learning in such a manner that the evaluation value r is increased, by using the Actor-Critic method. A learning method represented by Formula (1) is reinforcement learning that progresses in such a manner that a difference between a target and a state value decreases.

$${∇}_{θ} J ( θ ) = [ \text{?} ( \text{?} + γ \text{?} ( {S}_{t + 1} ) - \text{?} ( {S}_{t} ) ) {∇}_{θ} log {π}_{θ} ( \text{?} \vert {S}_{t} ) ] \qquad ( 1 )$$

$$\text{?} \text{indicates text missing or illegible when filed}$$

solid line: target
broken line: state value

A time t indicates an execution time of the previous image capturing in an image diagnosis examination including a plurality of times of image capturing. A time t+1 indicates an execution time of the next image capturing. Sₜ denotes subject information at the time t, i.e., subject information including previously-captured CT images. More specifically, the subject information Sₜ includes previously-captured CT images, and estimated images generated based on the previously-captured CT images. Sₜ₊₁ denotes subject information at the time t+1, i.e. subject information including a CT image to be captured the next image capturing. In addition, rₜ denotes the evaluation value r at the time t, “A” denotes an image capturing condition including an image capturing position, and γ is a variable indicating a discount ratio. The discount ratio indicates an unknown part, such as noise, for example, in a CT image to be captured next. In addition, θ is a network parameter, and ∇_{θ}J(θ) indicates a gradient in reinforcement learning.

The method is not limited to the Actor-Critic method, and another measure-based reinforcement learning may be employed. While, in the one embodiment, learning processing involving actual image capturing that is executed by the X-ray CT scanner 1a will be described, the learning function 454a may perform processing for the learning by simulation or phantom. The method is not limited to a method of executing image capturing and learning in real time, and offline reinforcement learning to execute learning a measure from image capturing data including a CT image captured in the past and an image capturing condition of the CT image may be used.

In response to a learned model generated by the learning function 454a receiving the input of subject information including a CT image and an image capturing condition of the CT image, the learned model outputs an image capturing condition under which the evaluation value r is increased, as an image capturing condition in the next image capturing. In other words, the learned model outputs an image capturing condition under which the image quality of a medical image improves, and the uncertainty of disease classification estimated to be obtained in a case where a disease is classified based on the medical image is highly likely decreased.

The learning function 454a according to the one embodiment may continue reinforcement learning not only in a learning phase before an operation start, but also in an operation phase.

Referring back to FIG. 1, the image capturing condition determination function 455a determines an image capturing condition in the next image capturing of the subject P, based on subject information including a CT image of the previous image capturing and a learned model generated by the learning function 454a. More specifically, the image capturing condition determination function 455a inputs the subject information including the CT image of the previous image capturing and an image capturing condition of the previous image capturing to the learned model, and obtains an image capturing condition under which the evaluation value r is most likely increased in the next image capturing.

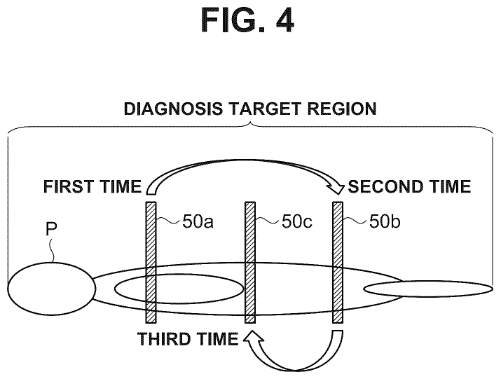

FIG. 4 is a diagram illustrating an example of the image capturing positions 50a to 50c in an image diagnosis examination including a plurality of times of image capturing according to the one embodiment. In an image diagnosis examination including a plurality of times of image capturing according to the one embodiment, the image capturing function 451a executes first-time image capturing under a predetermined image capturing condition. The predetermined image capturing condition may be automatically determined by an examination order or may be manually set by the user.

In the example illustrated in FIG. 4, in the first-time image capturing, an image of the image capturing position 50a specified by the predetermined image capturing condition is captured. Then, based on subject information including the CT image 51a obtained by the image capturing of the image capturing position 50a, the image capturing condition determination function 455a determines the image capturing position 50b within a diagnosis target region that is to be set in second-time image capturing. The image capturing condition in the first-time image capturing is an example of the first image capturing condition according to the one embodiment, and the CT image 51a is an example of the first medical image according to the one embodiment. An image capturing condition including the image capturing position 50b in the second-time image capturing is an example of a second image capturing condition according to the one embodiment. The CT image 51b captured under the second image capturing condition is an example of the second medical image according to the one embodiment. The image capturing condition determination function 455a according to the one embodiment determines an image capturing position included in an image capturing condition for the next image capturing from among previously-unimaged portions. For example, the image capturing position 50b included in the second image capturing condition is included in unimaged portions at a time point at which the first-time image capturing ends.

After the second-time image capturing, the image capturing condition determination function 455a determines the image capturing position 50c within the diagnosis target region that is to be set in third-time image capturing, based on subject information including the CT image 51b obtained by the image capturing of the image capturing position 50b. In this manner, the image capturing condition determination function 455a dynamically determines the next image capturing condition in accordance with the progress of image capturing.

An image capturing condition determined by the image capturing condition determination function 455a includes information regarding execution or non-execution of next image capturing, and an image capturing position of the subject P in a case where the next image capturing is to be executed. More specifically, in second-time and subsequent image capturing, the image capturing condition determination function 455a also determines whether to execute image capturing, in each image capturing. In the one embodiment, the X-ray CT scanner 1a does not fully capture images of the diagnosis target region (for example, the whole body of the subject P), and in a case where the image capturing condition determination function 455a determines that the next image capturing is not to be executed, although an unimaged portion exists within the diagnosis target region, the X-ray CT scanner 1a ends image capturing of the subject P.

The image capturing condition determination function 455a uses a learned model (image capturing measure model) trained by the learning function 454a as a determination method of the next image capturing condition. The image capturing condition determination function 455a inputs subject information including a previously-captured CT image to a learned model trained by the learning function 454a and determines an image capturing condition output by the learned model, under which the evaluation value r is most likely increased, as a next image capturing condition.

Determination processing to be executed by the image capturing condition determination function 455a is represented by the following Formula (2). Formula (2) represents an image capturing condition aₜ₊₁ to maximize an evaluation value at a next image capturing time (t+1) by the measure π, for example. In Formula (2), Sₜ denotes subject information at a time t. More specifically, the subject information Sₜ in Formula (2) includes a previously-captured CT image and an estimated image estimated from the CT image. In other words, Sₜ in Formula (2) indicates the state of the entire diagnosis target region of the subject P at the time t.

aₜ₊₁=argmax_{a∈A}π(a|sₜ) (2)

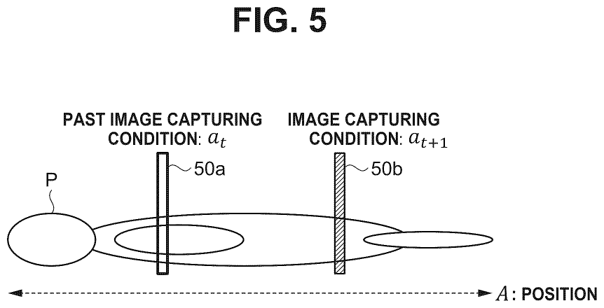

FIG. 5 is a diagram for an explanation of Formula (2) according to the one embodiment. As illustrated in FIG. 5, “A” indicates the entire range of the diagnosis target region of the subject P, i.e., the whole body of the subject P in the one embodiment, “a” indicates a position within the range A, and the image capturing position 50a included in an image capturing condition in the past image capturing, specifically, the previous image capturing, is denoted by aₜ, and the image capturing position 50b included in an image capturing condition to be obtained by the learned model, i.e., an image capturing condition in the next image capturing, is denoted by aₜ₊₁.

As a case where the learned model outputs a result indicating that next image capturing is not to be executed, i.e., end of image capturing, there is a state where the evaluation value r no longer improves even if the number of times of image capturing is further increased. Examples of the state where the evaluation value r no longer improves even if the number of times of image capturing is increased include a case where an evaluation value regarding the uncertainty of disease classification is no longer increased even if the number of times of image capturing is further increased. As another example of the state where the evaluation value r no longer improves even if the number of times of image capturing is further increased, there is a case where the evaluation value r is decreased by the above-described penalty, such as a case with an increase in the examination time or a case with an increase in an X-ray amount by further increasing the number of times of image capturing. In such a case, the image capturing condition determination function 455a determines the end of image capturing.

Referring back to FIG. 1, the display control function 456a controls the display 42a to display various screens and images. For example, the display control function 456a causes the display 42a to display a CT image captured by the image capturing function 451a. The display control function 456a may cause the display 42a to display the next image capturing condition determined by the image capturing condition determination function 455a. In the one embodiment, the image capturing function 451a may automatically start the next image capturing based on the next image capturing condition determined by the image capturing condition determination function 455a, or after the user checks the determined next image capturing condition on the display 42a, the next image capturing may be started based on an operation of the user.

The display control function 456a may cause the display 42a to display an operation screen on which the user can change the next image capturing condition determined by the image capturing condition determination function 455a. For example, instead of the image capturing function 451a automatically executing image capturing based on an image capturing condition, the user may manually set an image capturing condition of the X-ray CT scanner 1a in accordance with the setting of an image capturing condition displayed on the display 42a.

A procedure of the learning processing to be executed by the X-ray CT scanner 1a having the above-described configuration will be described.

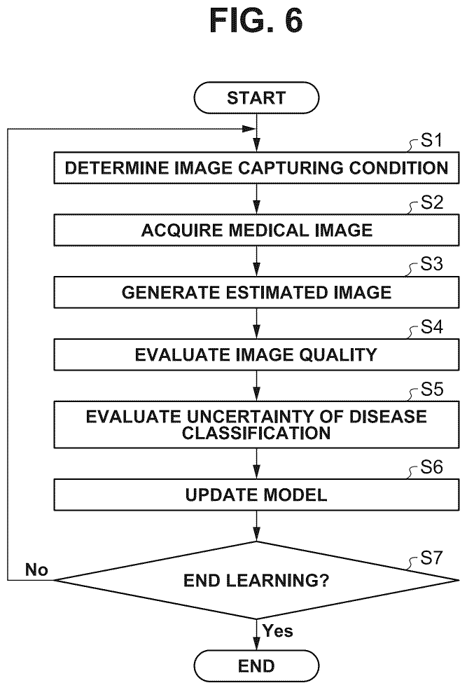

FIG. 6 is a flowchart illustrating an example of the procedure of the learning processing according to the one embodiment. Before execution of this flowchart, an examination order and medical care information of the subject P are acquired by the transmitting/receiving function 452a.

In step S1, an image capturing condition in the first-time image capturing is initially determined. In the first-time image capturing, image capturing may be executed based on a predetermined image capturing condition specified by the user, instead of an image capturing condition defined by the image capturing condition determination function 455a. The image capturing condition determination function 455a may determine an image capturing condition in the first-time image capturing, based on the examination order and the medical care information of the subject P. An image capturing condition in the learning processing is an example of an image capturing condition for learning according to the one embodiment.

In step S2, the image capturing function 451a acquires a medical image (the CT image 51) by executing the first-time image capturing under the determined image capturing condition. The CT image 51 is an example of a medical image for learning included in subject information for learning.

In step S3, the estimation function 453a generates the estimated image 61 for an unimaged portion within the diagnosis target region of the subject P that excludes the image capturing position 50 in the image capturing condition determined in step S1, based on the CT image 51 acquired in step S2. The estimated image 61 is an example of an estimated image for learning according to the one embodiment.

In step S4, the estimation function 453a evaluates the image quality of the CT image 51 and the estimated image 61. In the image quality evaluation, both the CT image 51 and the estimated image 61 may be targeted, or only the estimated image 61 may be targeted.

In step S5, the estimation function 453a evaluates the uncertainty of disease classification estimated to be obtained in a case where a disease is classified based on the CT image 51 and the estimated image 61. The estimation function 453a obtains the evaluation value r based on the evaluation executed in steps S4 and S5.

In step S6, the learning function 454a executes reinforcement learning in such a manner that the evaluation value r obtained by the estimation function 453a is increased, to update the model.

In step S7, the learning function 454a determines whether the learning ends. A learning end condition may be a case where learning converges or a case where the number of trials reaches a predetermined number of trials, for example. The predetermined number of trials may be arbitrarily determined by the user who executes learning.

In a case where learning is not to be ended (NO in step S7), the processing returns to step S1, in which the image capturing condition determination function 455a determines an image capturing condition in the second-time image capturing. As the second-time image capturing condition, an image capturing condition output by the learned model updated in step S6 is used. In this manner, while the learning is continued, the processing in steps S1 to S6 is repeatedly executed.

In a case where learning is to be ended (YES in step S7), the learning function 454a stores the learned model into the storage circuitry 41a, for example. Here, the processing of this flowchart ends.

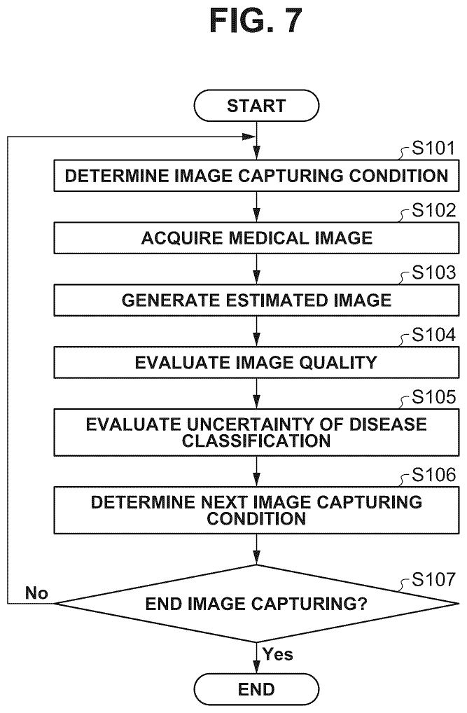

A procedure of image capturing processing to be executed during an operation after the learning will be described. FIG. 7 is a flowchart illustrating an example of a procedure of image capturing processing according to the one embodiment. This flowchart is executed in accordance with an examination order of an image diagnosis examination of the subject P, for example. Before the execution of this flowchart, the learning processing described with reference to FIG. 6 has been executed.

In step S101, an image capturing condition in the first-time image capturing is initially determined. The image capturing condition is an example of the first image capturing condition. Similarly to step S1 of FIG. 6, in the first-time image capturing, image capturing may be executed based on the predetermined image capturing condition specified by the user, instead of an image capturing condition defined by the image capturing condition determination function 455a. The image capturing condition determination function 455a may determine an image capturing condition in the first-time image capturing, based on the examination order and the medical care information of the subject P.

In step S102, the image capturing function 451a acquires a medical image (the CT image 51) by executing the first-time image capturing under the determined image capturing condition. The CT image 51 is an example of the first medical image.

In step S103, the estimation function 453a generates the estimated image 61 for an unimaged portion within the diagnosis target region of the subject P that excludes the image capturing position 50 in the image capturing condition in step S101, based on the CT image 51 acquired in step S102.

In step S104, the estimation function 453a evaluates the image quality of the CT image 51 acquired in step S102 and the estimated image 61 estimated in step S103. Similarly to step S4, in the image quality evaluation, both the CT image 51 and the estimated image 61 may be targeted, or only the estimated image 61 may be targeted.

In step S105, the estimation function 453a evaluates the uncertainty of disease classification estimated to be obtained in a case where a disease is classified based on the CT image 51 and the estimated image 61. The estimation function 453a obtains the evaluation value r based on the evaluation executed in steps S104 and S105.

Then, in step S106, the image capturing condition determination function 455a determines an image capturing condition in the second-time image capturing by inputting the image capturing condition determined in step S101, the CT image 51 acquired in step S102, and the estimated image 61 estimated in step S103, to a learned model. The image capturing condition determination function 455a may update the learned model using the evaluation value r obtained by the estimation function 453a. The learning during an operation is not essential. In a case where learning is not executed during an operation, the evaluation processing of image quality in step S104, the evaluation processing of the uncertainty of disease classification in step S105, and the calculation processing of the evaluation value r may be skipped.

In a case where the image capturing condition determined in step S106 does not indicate the end of image capturing (NO in step S107), the processing returns to step S101, and the image capturing condition determination function 455a determines an image capturing condition in the second-time image capturing.

In a case where the image capturing condition determined in step S106 indicates the end of image capturing (YES in step S107), the image diagnosis examination of the subject P ends. Here, the processing of this flowchart ends.

In this manner, the X-ray CT scanner 1a according to the one embodiment acquires the subject information regarding the subject P that includes the CT image 51 previously obtained by capturing the diagnosis target region of the subject P, and determines the image capturing condition for the next image capturing of the subject P based on the subject information and a learned model. The X-ray CT scanner 1a according to the one embodiment determines the image capturing condition for the next image capturing using the learned model that has learned evaluation information regarding a medical image and an image capturing condition in association. Thus, with the X-ray CT scanner 1a according to the one embodiment, during an image diagnosis examination in which two or more times of image capturing are consecutively executable, it is possible to automatically determine an appropriate image capturing condition for the next image capturing in accordance with a result of the previous image capturing. With this configuration, for example, it is possible to save the user's time and effort of considering and determining an image capturing condition, such as an image capturing range, based on an examination order which has been performed in conventional examinations.

An image capturing condition to be determined by the X-ray CT scanner 1a according to the one embodiment includes information regarding execution or non-execution of the next image capturing, and an image capturing position of the subject in a case where the next image capturing is to be executed. More specifically, in a case where non-execution of the next image capturing is output by the learned model, even if an unimaged portion exists within the diagnosis target region, the X-ray CT scanner 1a according to the one embodiment determines to end image capturing. Thus, with the X-ray CT scanner 1a according to the one embodiment, for example, an examination time and an amount of X-ray to be received by the subject P are reduced while the CT image 51 sufficient for diagnosis is captured, as compared with a case where images of the entire diagnosis target region of the subject P are captured.

The X-ray CT scanner 1a according to the one embodiment obtains evaluation information regarding the estimated image 61, based on the subject information obtained in the previous image capturing. The evaluation information includes at least either one of the first evaluation information regarding the image quality of the estimated image 61 and the second evaluation information regarding the uncertainty of disease classification estimated to be obtained in a case where a disease is classified based on the CT image 51 and the estimated image 61. Thus, with the X-ray CT scanner 1a according to the one embodiment, an image capturing condition is determined in consideration of both an image quality level and diagnosis accuracy as an image capturing condition for the next image capturing.

The estimated image according to the one embodiment is the medical image estimated to be captured in a case where an image of an unimaged portion of the diagnosis target region of the subject P is captured. The X-ray CT scanner 1a according to the one embodiment obtains the evaluation information based on the CT image 51 previously-captured and the estimated image 61. Thus, with the X-ray CT scanner 1a according to the one embodiment, the evaluation is executable in accordance with the state of the entire diagnosis target region of the subject P estimated based on the CT image 51 previously-captured and the estimated image 61 for the unimaged portion.

The X-ray CT scanner 1a according to the one embodiment determines an image capturing condition in the next image capturing by causing the model to execute learning in such a manner that evaluation information regarding the estimated image 61 for learning improves, based on an image capturing condition for learning, subject information for learning, and evaluation information for learning, and inputting the subject information including the CT image 51 captured in the previous image capturing and a previous image capturing condition to the learned model. Thus, with the X-ray CT scanner 1a according to the one embodiment, an image capturing condition for an unimaged portion is appropriately determined.

While, in the one embodiment, the CT image 51 is used as an example of the first medical image, the second medical image, and the medical image for learning, the first medical image, the second medical image, and the medical image for learning may be data based on which an image is to be generated, such as a signal detected by the X-ray detector 12 and detection data generated by the DAS 18, for example.

In an image diagnosis examination including a plurality of times of image capturing, the X-ray CT scanner 1a according to the one embodiment executes the first-time image capturing under the predetermined image capturing condition and determines an image capturing condition for the second-time or subsequent image capturing, by the estimation acquired based on the subject information. The X-ray CT scanner 1a may automatically determine an image capturing condition even for the first-time image capturing in the image diagnosis examination including a plurality of times of image capturing. For example, before the first-time image capturing is performed, the transmitting/receiving function 452a may acquire, as subject information, a CT image captured before the present image diagnosis examination including a plurality of times of image capturing, such as a CT image captured on the previous day, for example.

Medical image data captured by a different modality before the present image diagnosis examination including a plurality of times of image capturing may be acquired as the subject information. The subject information to be used for determining an image capturing condition in the first-time image capturing may be subject information not including a medical image. For example, the transmitting/receiving function 452a may acquire an examination order for the subject P or medical care information recorded on an electronic medical record, as the subject information to be used for determining an image capturing condition in the first-time image capturing. In this case, the estimation function 453a generates an estimated image based on the subject information and obtains evaluation information regarding the generated estimated image.

While, in the one embodiment, the estimation function 453a estimates the evaluation information regarding image quality of the estimated image and the evaluation information regarding the uncertainty of disease classification estimated to be obtained in a case where a disease is classified based on a CT image data included in subject information and an estimated image, the type of evaluation information is not limited to the types in the example. For example, the estimation function 453a may obtain at least either one of the evaluation information regarding image quality of the estimated image and the evaluation information regarding the uncertainty of disease classification estimated to be obtained in a case where a disease is classified based on the CT image data included in the subject information and the estimated image. Alternatively, the estimation function 453a may further obtain a different type of the evaluation information.

While, in the one embodiment, a learned model is stored in the storage circuitry 41a, for example, the learned model may be incorporated into the image capturing condition determination function 455a. The learned model may be stored into a storage device disposed outside the X-ray CT scanner 1a and may be read at the time of processing.

### Another Embodiment

In the one embodiment, the X-ray CT scanner 1a has been described as an example of the medical image diagnosis apparatus. In another embodiment, a magnetic resonance imaging (MRI) apparatus will be described as an example of the medical image diagnosis apparatus.

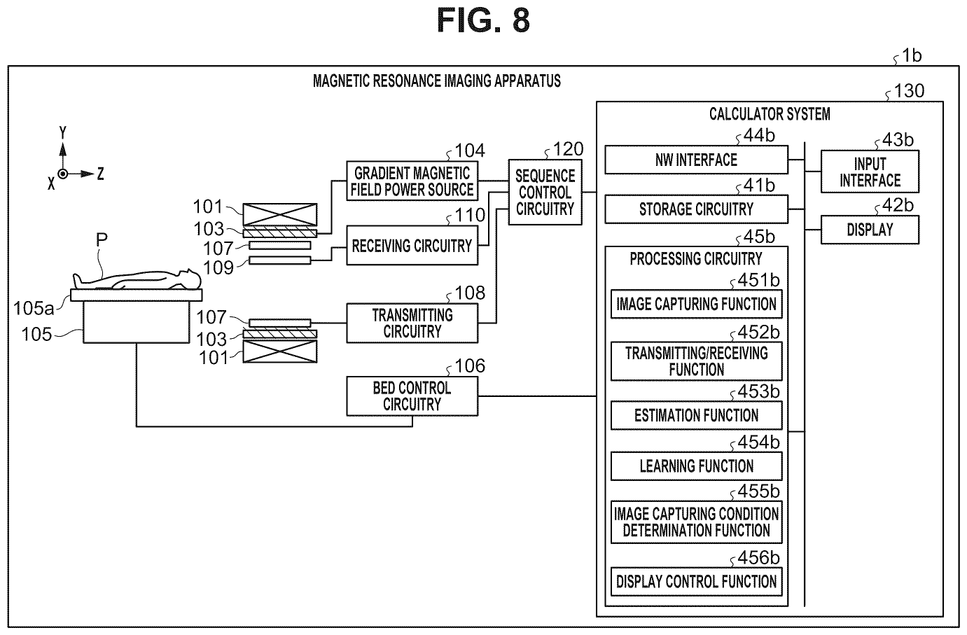

FIG. 8 is a diagram illustrating an example of a configuration of a magnetic resonance imaging apparatus 1b according to the another embodiment. As illustrated in FIG. 8, the magnetic resonance imaging apparatus 1b includes a magnetostatic field magnet 101, a magnetostatic field power source (not illustrated), a gradient magnetic field coil 103, a gradient magnetic field power source 104, a bed 105, a bed control circuitry 106, a transmitting coil 107, a transmitting circuitry 108, a receiving coil 109, a receiving circuitry 110, a sequence control circuitry 120, and a calculator system 130. The configuration of the magnetic resonance imaging apparatus 1b illustrated in FIG. 8 is an example, and the configuration is not limited to this.

The configuration illustrated in FIG. 8 is merely an example. For example, components in the sequence control circuitry 120 and the calculator system 130 may be integrated or separated as appropriate. A subject P is not included in the magnetic resonance imaging apparatus 1b.

An X-axis, Y-axis, and Z-axis illustrated in FIG. 8 are included in an apparatus coordinate system unique to the magnetic resonance imaging apparatus 1b. For example, the Z-axis direction coincides with an axis direction of a cylinder of the gradient magnetic field coil 103 and is set along a magnetic flux of a magnetostatic field that is generated by the magnetostatic field magnet 101. The Z-axis direction coincides with a longer direction of the bed 105 and also coincides with a cephalocaudal direction of the subject P laid on the bed 105. The X-axis direction is set in a horizontal direction orthogonal to the Z-axis direction. The Y-axis direction is set in a vertical direction orthogonal to the Z-axis direction.

The magnetostatic field magnet 101 is a magnet having a hollow approximately-cylindrical shape and generates a magnetostatic field in the internal space. The magnetostatic field magnet 101 is a superconducting magnet, for example, and is excited in response to receipt of current supplied from the magnetostatic field power source. The magnetostatic field power source supplies current to the magnetostatic field magnet 101. Alternatively, the magnetostatic field magnet 101 may be a permanent magnet. In this case, the magnetostatic field power source may be omitted from the magnetic resonance imaging apparatus 1b. The magnetostatic field power source may be disposed separately from the magnetic resonance imaging apparatus 1b.

The gradient magnetic field coil 103 is a coil having a hollow approximately-cylindrical shape and disposed on the inside of the magnetostatic field magnet 101. The gradient magnetic field coil 103 is a combination of three coils respectively corresponding to X, Y, and Z axes orthogonal to each other, and these three coils individually receive the current supplied from the gradient magnetic field power source 104 and generate a gradient magnetic field changing in magnetic field intensity along the X, Y, and Z axes. Under the control of the sequence control circuitry 120, the gradient magnetic field power source 104 supplies current to the gradient magnetic field coil 103.

The bed 105 includes a top panel 105a on which the subject P is laid, and under the control executed by the bed control circuitry 106, the top panel 105a is inserted into an image capturing port in a state in which the subject P, such as a patient, is laid thereon. Under the control executed by the calculator system 130, the bed control circuitry 106 drives the bed 105 to move the top panel 105a in the longer direction and the up-down direction.

With application of a high-frequency magnetic field, the transmitting coil 107 excites a certain region of the subject P. The transmitting coil 107 is a whole body type coil surrounding the whole body of the subject P, for example. In response to receipt of the supply of a radio frequency (RF) pulse from the transmitting circuitry 108, the transmitting coil 107 generates a high-frequency magnetic field and applies the high-frequency magnetic field to the subject P. Under the control of the sequence control circuitry 120, the transmitting circuitry 108 supplies the RF pulse to the transmitting coil 107.

The receiving coil 109 is disposed on the inside of the gradient magnetic field coil 103, and receives a magnetic resonance signal (hereinafter, will be referred to as an MR signal) transmitted from the subject P due to the influence of the high-frequency magnetic field. In response to receipt of the MR signal, The receiving coil 109 outputs the received MR signal to the receiving circuitry 110.

While, FIG. 8 illustrates a configuration in which the receiving coil 109 is disposed separately from the transmitting coil 107, this is an example, and the configuration is not limited to this configuration. For example, a configuration in which the receiving coil 109 also serves as the transmitting coil 107 may be employed.

The receiving circuitry 110 performs analog-to-digital (AD) conversion of an analog MR signal output from the receiving coil 109 and generates MR data. The receiving circuitry 110 transmits the generated MR data to the sequence control circuitry 120. The AD conversion may be performed within the receiving coil 109. Aside from AD conversion, the receiving circuitry 110 performs certain signal processing.

The sequence control circuitry 120 performs image capturing of the subject P by driving the gradient magnetic field power source 104, the transmitting circuitry 108, and the receiving circuitry 110, based on sequence information transmitted from the calculator system 130.

Here, the sequence information is information defining a procedure for image capturing. The sequence information defines the intensity of current to be supplied by the gradient magnetic field power source 104 to the gradient magnetic field coil 103, a timing at which current is supplied, the intensity of the RF pulse to be supplied by the transmitting circuitry 108 to the transmitting coil 107, a timing at which the RF pulse is applied, and a timing at which the receiving circuitry 110 detects the MR signal. The sequence information varies in accordance with the range of a region of the body of the subject P that serves as an image capturing target.

The sequence control circuitry 120 may be implemented by a processor or may be implemented by software and hardware in combination.

In response to receipt of MR data from the receiving circuitry 110 as a result of capturing an image of the subject P by driving the gradient magnetic field power source 104, the transmitting circuitry 108, and the receiving circuitry 110, the sequence control circuitry 120 transfers the received MR data to the calculator system 130.

The calculator system 130 performs control of entire operation of the magnetic resonance imaging apparatus 1b and generation of MR images. The calculator system 130 performs control of entire operation of the magnetic resonance imaging apparatus 1b and generation of MR images. As illustrated in FIG. 8, the calculator system 130 includes a NW interface 44b, a storage circuitry 41b, a processing circuitry 45b, an input interface 43b, and a display 42b.

Similarly to the X-ray CT scanner 1a according to the one embodiment that has been described with reference to FIG. 1, the magnetic resonance imaging apparatus 1b may be connected with the electronic medical record system 2 in such a manner that communication is performed with each other.

The processing circuitry 45b controls entire operation of the magnetic resonance imaging apparatus 1b. For example, the processing circuitry 45b includes an image capturing function 451b, a transmitting/receiving function 452b, an estimation function 453b, a learning function 454b, an image capturing condition determination function 455b, and a display control function 456b. Here, for example, processing functions that are executed by the image capturing function 451b, the transmitting/receiving function 452b, the estimation function 453b, the learning function 454b, the image capturing condition determination function 455b, and the display control function 456b, which are components of the processing circuitry 45b illustrated in FIG. 8, are recorded in the storage circuitry 41b in the form of computer-executable programs. The processing circuitry 45b is a processor, for example, and reads out each program from the storage circuitry 41b and implements a function corresponding to the program, by executing the program. In other words, the processing circuitry 45b in a state in which each program is read out has a corresponding function illustrated in the processing circuitry 45b in FIG. 8. The image capturing function 451b is an example of an imaging unit. The transmitting/receiving function 452b is an example of a transmitting/receiving unit and an output unit. The image capturing function 451b and the transmitting/receiving function 452b serve as an example of an acquisition unit. The estimation function 453b is an example of an estimation unit. The learning function 454b is an example of a learning unit. The image capturing condition determination function 455b is an example of an image capturing condition determination unit. The display control function 456b is an example of a display control unit and an output unit.

While FIG. 8 illustrates a case where the image capturing function 451b, the transmitting/receiving function 452b, the estimation function 453b, the learning function 454b, the image capturing condition determination function 455b, and the display control function 456b are implemented by a single processing circuitry, i.e., the processing circuitry 45b, the another embodiment is not limited to this. For example, the processing circuitry 45b may include a combination of a plurality of independent processors, and each processor may implement the corresponding processing function by executing the corresponding program. Each processing function included in the processing circuitry 45b may be implemented by being appropriately allocated to a plurality of processing circuitries or integrated into a single processing circuitry.

The image capturing function 451b executes image capturing of a magnetic resonance image by controlling various components of the magnetic resonance imaging apparatus 1b, based on an image capturing condition. For example, the image capturing function 451b executes generation of the sequence information, the collection of MR data, generation of k-space data, and generation of a magnetic resonance image.

More specifically, the image capturing function 451b generates the sequence information specifying a procedure of image capturing, in accordance with the type of a magnetic resonance image to be captured and an image capturing target region, and transmits the generated sequence information to the sequence control circuitry 120 via the NW interface 44b. The sequence control circuitry 120 executes various pulse sequences based on the sequence information generated by the image capturing function 451b.

The image capturing function 451b collects MR data converted from an MR signal transmitted from the subject P by execution of the various pulse sequences, from the sequence control circuitry 120 via the NW interface 44b. The image capturing function 451b arranges the collected MR data in accordance with a phase encoding amount or a frequency encoding amount induced by a gradient magnetic field. The MR data arranged in the k-space will be referred to as k-space data. The k-space data is stored into the storage circuitry 41b.

The image capturing function 451b generates a magnetic resonance image based on the k-space data stored in the storage circuitry 41b. For example, by reconstruction processing such as Fourier transformation on the k-space data, the image capturing function 451b generates a magnetic resonance image. The image capturing function 451b stores the generated magnetic resonance image into the storage circuitry 41b, for example. The magnetic resonance image captured by the image capturing function 451b is an example of a first medical image, a second medical image, and a medical image for learning according to the another embodiment.

An image capturing condition according to the another embodiment includes at least an image capturing position and the type of a magnetic resonance image. The image capturing condition may include an image capturing region, a repetition time (TR), an echo time (TE), the number of slices, an image capturing direction, and a slice thickness. The type of the magnetic resonance image includes, for example, a T1 weighted image (T1WI), a T2 weighted image (T2WI), and a fluid attenuated inversion recovery (FLAIR) image.

Subject information according to the another embodiment includes at least a magnetic resonance image captured by the magnetic resonance imaging apparatus 1b.

Similarly to the transmitting/receiving function 452a according to the one embodiment, the transmitting/receiving function 452b may acquire the subject information regarding the subject P except for magnetic resonance images, from the electronic medical record system 2 or a different apparatus via the network N and the NW interface 44b.

Based on the subject information, the estimation function 453b obtains evaluation information regarding an estimated image estimated to be obtained by image capturing of a diagnosis target region of the subject P under an image capturing condition different from an image capturing condition used when a magnetic resonance image included in the subject information has been captured.

In magnetic resonance imaging, because image capturing is sometimes performed on the same image capturing position by using different pulse sequences, an image capturing position included in the next image capturing condition sometimes corresponds not only to an unimaged portion within a diagnosis target region of the subject P but also coincides with the previously-imaged portion. Under the different image capturing condition, at least either one of an image capturing position, an image capturing direction, or the type of the image is different. The image capturing condition does not have to directly specify the type of image, but may indirectly specify the type of image directly using various parameters of the pulse sequence. Examples of image capturing directions in magnetic resonance imaging include a coronal plane direction, a sagittal plane direction, and an axial plane direction.

As a method of obtaining the evaluation information, the method described in the one embodiment is usable.

By using the method described in the one embodiment, the learning function 454b causes a model to execute learning while associating evaluation information regarding a medical image (for example, magnetic resonance image) and an image capturing condition in such a manner that evaluation information (for example, the evaluation value r) is increased, and generates a learned model.

The image capturing condition determination function 455b determines an image capturing condition in the next image capturing of the subject P, based on subject information including a magnetic resonance image of the previous image capturing and the learned model generated by the learning function 454b. As a method of determining an image capturing condition, the method described in the one embodiment is usable.

The display control function 456b controls the display 42b to display various screens and images.

A procedure of image capturing processing to be executed by the magnetic resonance imaging apparatus 1b according to the another embodiment with the above-described configuration will be described.

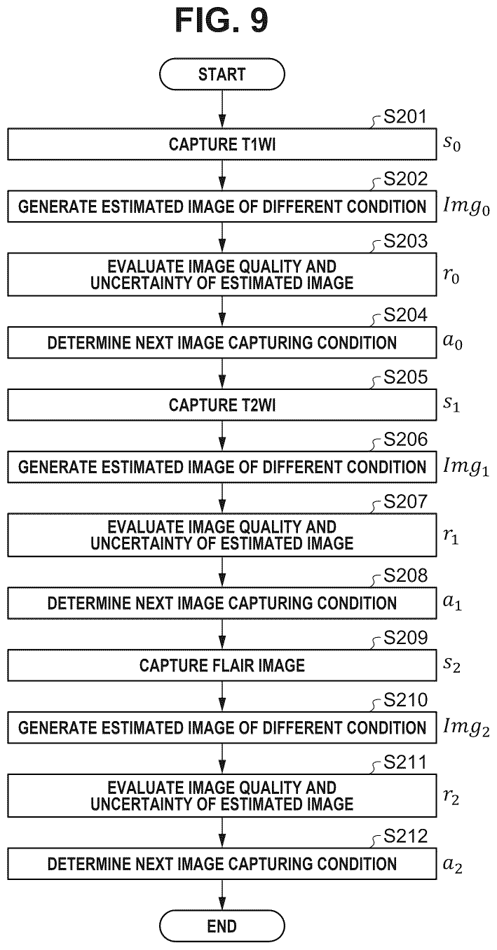

FIG. 9 is a flowchart illustrating an example of a procedure of image capturing processing according to the another embodiment. The image capturing processing according to the another embodiment is executed as an MRI sequence.

An image capturing condition in first-time image capturing is initially determined, and in step S201, the image capturing function 451a acquires a magnetic resonance image by the first-time image capturing executed under the determined image capturing condition. The processing corresponds to the processing in steps S101 and S102 in the procedure of the image capturing processing according to the one embodiment that has been described with reference to FIG. 7. In the example illustrated in FIG. 9, the T1WI is specified as the type of image in a first-time image capturing condition. Similarly to the one embodiment, in the first-time image capturing, image capturing may be executed based on a predetermined image capturing condition specified by the user, instead of an image capturing condition defined by the image capturing condition determination function 455b. In FIG. 9, a magnetic resonance image captured in the first-time image capturing is represented as subject information S₀.

In step S202, the estimation function 453b generates an estimated image based on the magnetic resonance image (subject information S₀) acquired in step S201. The processing corresponds to the processing in step S103 in the flow of image capturing processing according to the one embodiment that has been described with reference to FIG. 7. In FIG. 9, an estimated image that is generated based on the magnetic resonance image captured in the first-time image capturing is represented as an estimated image Img₀. The estimated image Img₀ differs in image capturing position or image type from the magnetic resonance image (subject information S₀). In a case where the image type is different, image capturing positions of the estimated image Img₀ and the subject information S₀ may be coincide with each other. That is to say, an image capturing position of an estimated image according to the another embodiment is not limited to an unimaged portion.

In step S203, the estimation function 453b evaluates image quality of the estimated image Img₀ and the magnetic resonance image (subject information S₀), and the uncertainty of disease classification estimated to be obtained in a case where a disease is classified based on the estimated image Img₀ and the magnetic resonance image (subject information S₀). The processing corresponds to the processing in steps S104 and S105 in the procedure of the image capturing processing according to the one embodiment described with reference to FIG. 7. In FIG. 9, an evaluation value of the estimated image Img₀ is denoted by r₀.

In step S204, the image capturing condition determination function 455b determines an image capturing condition in second-time image capturing by inputting the image capturing condition of the image capturing executed in step S201 and the magnetic resonance image (subject information S₀) acquired in step S201, to the learned model. The processing corresponds to the processing in step S106 in the procedure of the image capturing processing according to the one embodiment described with reference to FIG. 7. The image capturing condition determination function 455b may update the learned model by using the evaluation value r that has been obtained by the estimation function 453b. An execution condition of the second-time image capturing is represented as a₀.

In the example illustrated in FIG. 9, the T2WI is specified as the type of image in an image capturing condition of the second-time image capturing. In this case, in step S205, the image capturing function 451a captures the T2WI. In FIG. 9, subject information including a magnetic resonance image captured in the second-time image capturing is represented as subject information S₁.

In step S206, the estimation function 453b generates an estimated image Img₁ based on the magnetic resonance image (subject information S₁) acquired in step S205.

In step S207, the estimation function 453b obtains an evaluation value r₁ for evaluating image quality of the estimated image Img₁ and the magnetic resonance image (subject information S₁), and the uncertainty of disease classification estimated to be obtained in a case where a disease is classified based on the estimated image Img₁ and the magnetic resonance image (subject information S₁).

In step S208, the image capturing condition determination function 455b determines an image capturing condition a₁ of third-time image capturing by inputting the image capturing condition of image capturing performed in step S205 and the magnetic resonance image (subject information S₁) acquired in step S205, to a learned model.

In the example illustrated in FIG. 9, the FLAIR image is specified as the type of image in an image capturing condition of the third-time image capturing. The processing from FLAIR image capturing in step S209 to evaluation processing in step S211 is executed in a procedure similar to that of the processing in steps S205 to S207. In the example illustrated in FIG. 9, in step S212, the image capturing condition determination function 455b determines to end image capturing as the next image capturing condition. Here, the processing of this flowchart ends. In a case where the image capturing condition determination function 455b determines to further execute image capturing of an image of a different type, or image capturing at a different image capturing position, the image capturing processing continues.

In this manner, in the another embodiment, also in the magnetic resonance imaging apparatus 1b similarly to the X-ray CT scanner 1a according to the one embodiment, during the image diagnosis examination in which two or more times of image capturing are consecutively executable, an appropriate image capturing condition is automatically determined for the next image capturing in accordance with a result of the previous image capturing.

### Yet Another Embodiment

In yet another embodiment, ultrasound diagnosis apparatus will be described as an example of a medical image diagnosis apparatus.

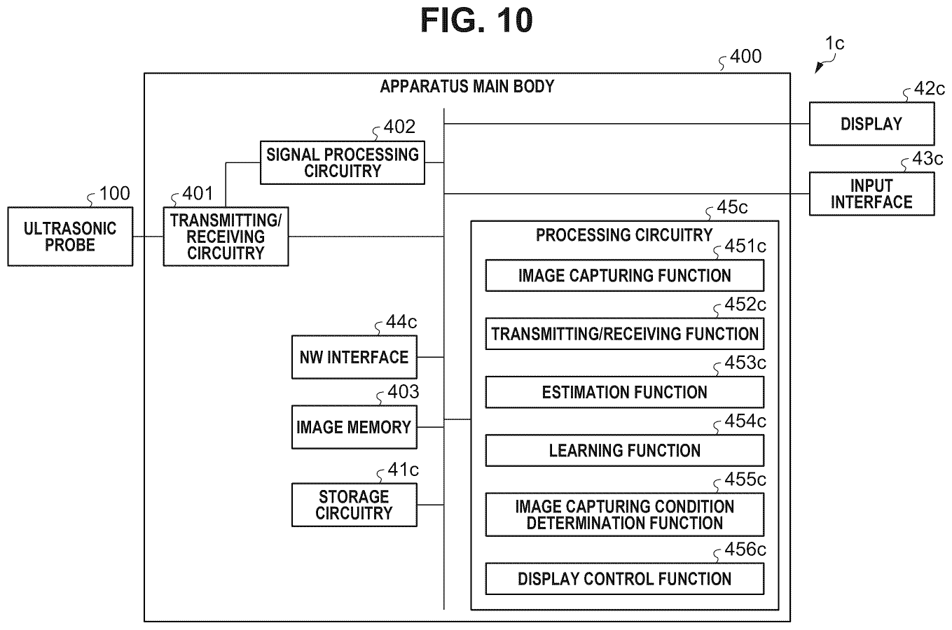

FIG. 10 is a diagram illustrating an example of a configuration of an ultrasound diagnosis apparatus 1c according to the yet another embodiment. As illustrated in FIG. 10, the ultrasound diagnosis apparatus 1c according to the yet another embodiment includes an ultrasonic probe 100, a display 42c, an input interface 43c, and an apparatus main body 400, and the ultrasonic probe 100, the display 42c, and the input interface 43c are connected with the apparatus main body 400 in such a manner that communication is performed with each other. The configuration of the ultrasound diagnosis apparatus 1c illustrated in FIG. 10 is an example, and the configuration is not limited to this.

The ultrasonic probe 100 is operated by the user and transmits ultrasonic waves to the subject P. Specifically, the ultrasonic probe 100 includes a plurality of piezoelectric vibrators, and the plurality of piezoelectric vibrators generates ultrasonic waves based on a drive signal supplied from a transmitting/receiving circuitry 401. The ultrasonic probe 100 receives waves reflected from the subject P and converts the reflected waves into electric signals. The ultrasonic probe 100 includes a matching layer disposed on the piezoelectric vibrator and a backing material that prevents propagation of ultrasonic waves backward from the piezoelectric vibrator. The ultrasonic probe 100 is detachably connected with the apparatus main body 400.

When ultrasonic waves are transmitted from the ultrasonic probe 100 to the subject P, the transmitted ultrasonic waves are sequentially reflected on an acoustic impedance discontinuous surface in a body tissue of the subject P and received as reflected wave signals by the plurality of piezoelectric vibrators included in the ultrasonic probe 100. The amplitude of the reflected wave signals to be received depends on a difference in acoustic impedance on the discontinuous surface on which ultrasonic waves are reflected. A reflected wave signal to be received in a case where a transmitted ultrasonic wave pulse is reflected by a moving blood flow or a surface of a heart wall receives frequency shift modulation due to a Doppler effect depending on a speed component in an ultrasonic wave transmission direction of a movable body.

The ultrasonic probe 100 may be a one-dimensional ultrasonic probe in which a plurality of piezoelectric vibrators are arranged in one line, or may be an ultrasonic probe that mechanically swings a plurality of piezoelectric vibrators of one-dimensional ultrasonic probe, or a two-dimensional ultrasonic probe in which a plurality of piezoelectric vibrators are two-dimensionally arranged like a grid.

The display 42c displays a graphical user interface (GUI) that is for a user of the ultrasound diagnosis apparatus 1c to input various setting requests by using the input interface 43c, and an ultrasonic image generated by the apparatus main body 400. The display 42c displays various messages or display information to notify the user of a processing status or a processing result of the apparatus main body 400. The display 42c includes a speaker to output voice.

The input interface 43c is operated to perform a setting of a predetermined position (for example, position of region of interest (ROI), etc.) and is implemented by, for example, a touch-pad that performs an input operation based on a touch on a trackball, a switch button, a mouse, a keyboard, or an operation surface, a touch monitor in which a display screen and a touch-pad are integrated, a contactless input circuitry that uses an optical sensor, and a voice input circuitry. The input interface 43c is connected to a processing circuitry 45c to be described below, converts an input operation received from the user into an electric signal, and outputs the electric signal to the processing circuitry 45c. In this specification, the input interface 43c is not limited to an interface that includes a physical operational component, such as a mouse and a keyboard. For example, a processing circuitry that receives an electric signal corresponding to an input operation from an external input device disposed separately from an apparatus and outputs the electric signal to the processing circuitry 45c is also included in the examples of the input interface 43c.

The apparatus main body 400 is an apparatus that generates an ultrasonic image based on a reflected wave signal received by the ultrasonic probe 100, and as illustrated in FIG. 10, includes a transmitting/receiving circuitry 401, a signal processing circuitry 402, an image memory 403, a storage circuitry 41c, a NW interface 44c, and the processing circuitry 45c.

Similarly to the X-ray CT scanner 1a according to the one embodiment described with reference to FIG. 1, the ultrasound diagnosis apparatus 1c may connect with the electronic medical record system 2 in such a manner that communication is performed with each other.

The processing circuitry 45c controls entire operation of the ultrasound diagnosis apparatus 1c. For example, the processing circuitry 45c includes an image capturing function 451c, a transmitting/receiving function 452c, an estimation function 453c, a learning function 454c, an image capturing condition determination function 455c, and a display control function 456c. Here, for example, processing functions to be executed by the image capturing function 451c, the transmitting/receiving function 452c, the estimation function 453c, the learning function 454c, the image capturing condition determination function 455c, and the display control function 456c, which are components of the processing circuitry 45c illustrated in FIG. 10, are recorded in the storage circuitry 41c in the form of computer-executable programs. The processing circuitry 45c is a processor, for example, and reads out each program from the storage circuitry 41c and implements a function corresponding to the program, by executing the program. In other words, the processing circuitry 45c in a state in which each program is read out has a corresponding function illustrated in the processing circuitry 45c in FIG. 10. The image capturing function 451c is an example of an imaging unit. The transmitting/receiving function 452c is an example of a transmitting/receiving unit and an output unit. The image capturing function 451c and the transmitting/receiving function 452c serve as an example of an acquisition unit. The estimation function 453c is an example of an estimation unit. The learning function 454c is an example of a learning unit. The image capturing condition determination function 455c is an example of an image capturing condition determination unit. The display control function 456c is an example of a display control unit and an output unit.

While FIG. 10 illustrates a case where the image capturing function 451c, the transmitting/receiving function 452c, the estimation function 453c, the learning function 454c, the image capturing condition determination function 455c, and the display control function 456c are implemented by a single processing circuitry, i.e., the processing circuitry 45c, the yet another embodiment is not limited to this. For example, the processing circuitry 45c may be a combination of a plurality of independent processors, and each processor may implement a corresponding processing function by executing a corresponding program. Each processing function included in the processing circuitry 45c may be implemented by being appropriately allocated to a plurality of processing circuitries or integrated into a single processing circuitry.

An image capturing condition according to the yet another embodiment includes probe information related to an operation of the ultrasonic probe 100, and a dynamic range, a gain, a focal spot, and a depth related to capturing of ultrasonic images. The probe information includes an angle and a position of the ultrasonic probe 100. The angle and the position of the ultrasonic probe 100 serve as an example of an image capturing position according to the yet another embodiment.

The image capturing function 451c captures an ultrasonic image by controlling various components of the ultrasound diagnosis apparatus 1c. The ultrasonic image captured by the image capturing function 451c is an example of a first medical image, a second medical image, and a medical image for learning according to the yet another embodiment.

Similarly to the transmitting/receiving function 452a according to the one embodiment, the transmitting/receiving function 452c may acquire subject information regarding the subject P except for ultrasonic images, from the electronic medical record system 2 or a different apparatus via the network N and the NW interface 44c.

Based on the subject information, the estimation function 453c obtains evaluation information regarding an estimated image estimated to be obtained by the image capturing of a diagnosis target region of the subject P under an image capturing condition different from an image capturing condition used when an ultrasonic image included in the subject information has been captured.

In ultrasonic image diagnosis, because images of different types are sometimes captured at the same image capturing position, an image capturing position included in the next image capturing condition sometimes corresponds not only to an unimaged portion within a diagnosis target region of the subject P but coincides also with a previously-imaged portion. Under the different image capturing condition, at least either one of an image capturing position or the type of the image is different. The image capturing condition does not have to directly specify the type of image, but may indirectly specify the type of image directly using various parameters.

As a method of obtaining the evaluation information, the method described in the one embodiment is usable.

By using the method described in the one embodiment, the learning function 454c generates a learned model by causing a model to execute learning while associating evaluation information regarding a medical image (for example, ultrasonic image) and an image capturing condition in such a manner that evaluation information (for example, the evaluation value r) is increased.

The image capturing condition determination function 455c determines an image capturing condition in the next image capturing of the subject P, based on the subject information including an ultrasonic image of the previous image capturing and the learned model generated by the learning function 454c. As a method of determining an image capturing condition, the method described in the one embodiment is usable.

The display control function 456c controls the display 42c to display various screens and images. The display control function 456c may cause the display 42c to display the probe information in the next image capturing that has been determined by the image capturing condition determination function 455c. Based on the probe information displayed on the display 42c, the user presses the ultrasonic probe 100 against the subject P.

In a case where the ultrasound diagnosis apparatus 1c is a fully automatic ultrasound diagnosis apparatus, the ultrasonic probe 100 may be automatically operated. In this case, the image capturing function 451c controls the angle and the position of the ultrasonic probe 100 based on the probe information determined by the image capturing condition determination function 455c.

In this manner in the yet another embodiment, also in the ultrasound diagnosis apparatus 1c similarly to the X-ray CT scanner 1a according to the one embodiment, during an image diagnosis examination in which two or more times of image capturing are consecutively executable, an appropriate image capturing condition for the next image capturing is automatically determined in accordance with a result of the previous image capturing.

In the above-described embodiments, the X-ray CT scanner 1a, the magnetic resonance imaging apparatus 1b, and the ultrasound diagnosis apparatus 1c are used as examples of the medical image diagnosis apparatus, but the medical image diagnosis apparatus is not limited to these, and may be another modality.

### Yet Another Embodiment

While, in the above-described embodiments, a medical image diagnosis apparatus executes processing, in yet another embodiment, an example in which processing is executed by a medical image processing apparatus will be described.

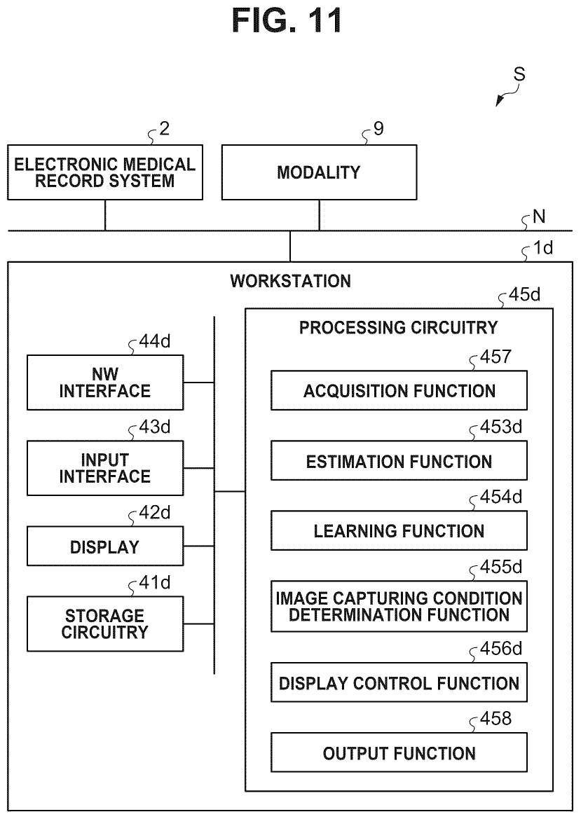

FIG. 11 is a diagram illustrating an example of a configuration of a workstation 1d according to the yet another embodiment. The workstation Id is an example of the medical image processing apparatus according to the yet another embodiment. The workstation 1d connects with the electronic medical record system 2 and a modality 9 via a network N, for example, in such a manner that communication is performed with each other. While, in FIG. 11, the workstation 1d connects with a single modality, i.e., the modality 9, the workstation 1d may connect with a plurality of modalities 9.

As illustrated in FIG. 11, the workstation 1d includes a storage circuitry 41d, a display 42d, an input interface 43d, a NW interface 44d, and a processing circuitry 45d. The configuration of the workstation 1d illustrated in FIG. 11 is an example, and the configuration is not limited to this.

The processing circuitry 45d controls entire operation of the workstation 1d. For example, the processing circuitry 45d includes an acquisition function 457, an estimation function 453d, a learning function 454d, an image capturing condition determination function 455d, a display control function 456d, and an output function 458. Here, for example, processing functions to be executed by the acquisition function 457, the estimation function 453d, the learning function 454d, the image capturing condition determination function 455d, the display control function 456d, and the output function 458, which are components of the processing circuitry 45d illustrated in FIG. 11, are recorded in the storage circuitry 41d in the form of computer-executable programs. The processing circuitry 45d is a processor, for example, and reads out each program from the storage circuitry 41d and implements the corresponding function corresponding to the program, by executing the program. In other words, the processing circuitry 45d in a state in which each program is read out has a corresponding function illustrated in the processing circuitry 45d in FIG. 11. The acquisition function 457 is an example of an acquisition unit. The estimation function 453d is an example of an estimation unit. The learning function 454d is an example of a learning unit. The image capturing condition determination function 455d is an example of an image capturing condition determination unit. The display control function 456d is an example of a display control unit. The output function 458 is an example of an output unit.

While FIG. 11 illustrates a case where the acquisition function 457, the estimation function 453d, the learning function 454d, the image capturing condition determination function 455d, the display control function 456d, and the output function 458 are implemented by a single processing circuitry, i.e., the processing circuitry 45d, the yet another embodiment is not limited to this. For example, the processing circuitry 45d may be a combination of a plurality of independent processors, and each processor may implement a corresponding processing function by executing a corresponding program. Each processing function included in the processing circuitry 45d may be implemented by being appropriately allocated to a plurality of processing circuitries or integrated into a single processing circuitry.

An image capturing condition according to the yet another embodiment may be changed in accordance with the type of the modality 9 serving as the target.

An ultrasonic image captured by the modality 9 is an example of a first medical image, a second medical image, and a medical image for learning according to the yet another embodiment.

From the modality 9, the acquisition function 457 acquires subject information regarding the subject P that includes a medical image obtained by capturing an image of a diagnosis target region of the subject P. Similarly to the transmitting/receiving function 452a according to the one embodiment, the acquisition function 457 acquires subject information regarding the subject P except for medical images, from the electronic medical record system 2 or a different apparatus.

The processing similar to the processing according to the one embodiment, for example, is applicable to the processing that is executed by the estimation function 453d, the learning function 454d, the image capturing condition determination function 455d, and the display control function 456d. The processing similar to the processing according to the above-described another embodiment or the above-described yet another embodiment may be applied in accordance with the type of the modality 9.

The output function 458 transmits an image capturing condition determined by the image capturing condition determination function 455d to the modality 9. With this configuration, image capturing that is based on the determined image capturing condition is executed by the modality 9. An output method of the image capturing condition is not limited to this, and the display control function 456d may display an image capturing condition on the display 42d. In this case, the user may manually set an image capturing condition of the modality 9 in accordance with the setting of an image capturing condition displayed on the display 42d.

In this manner, in the yet another embodiment, not in the modality 9 but in the workstation 1d as well, during the image diagnosis examination in which the modality 9 consecutively execute image capturing two or more times, an appropriate image capturing condition for the next image capturing is automatically determined in accordance with a result of the previous image capturing. With the workstation Id according to the yet another embodiment, because the function of determining an image capturing condition may be omitted from the modality 9, the modality 9 of an existing configuration is utilized.

The medical image processing apparatus is not limited to the workstation 1d and is any information processing apparatus as long as the information processing apparatus is capable of communicating with the modality 9. For example, the medical image processing apparatus may be various servers or a personal computer (PC). The functions of the workstation 1d according to the yet another embodiment may be allocated to and executed by a plurality of PCs.

While in the above-described embodiments, processing in a learning phase and processing in an operation phase are executed by one apparatus, the processing in the learning phase and the processing in the operation phase may be executed by different apparatuses. For example, the processing in the learning phase may be executed by a server or a PC, and the processing in the operation phase may be executed by various modalities or the workstation.

In place of or in addition to the model of deep learning or machine learning in the above-described embodiments, a mathematical model, a look-up table, or a database may also be employed.

Various types of data described in this specification are typically digital data.

According to at least one of the above-described embodiments, during an image diagnosis examination in which two or more times of image capturing are consecutively executable, an appropriate image capturing condition for the next image capturing is automatically determined in accordance with a result of the previous image capturing.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A medical image diagnosis apparatus comprising:
a medical scanner configured to perform a scan on a subject to obtain an image, processing circuitry configured to:
acquire subject information regarding the subject that includes a first medical image obtained by capturing an image of a diagnosis target region of the subject under a first image capturing condition,
determine a second image capturing condition to capture a second medical image of the subject, based on the subject information and a learned model obtained by executing learning while associating evaluation information regarding a medical image and an image capturing condition,
control the medical scanner to perform the scan on the subject by using the medical scanner with the determined second image capturing condition, and
obtain a medical image of the subject from the medical scanner.

2. The medical image diagnosis apparatus according to claim 1, wherein the second image capturing condition includes information regarding execution or non-execution of next image capturing, and an image capturing position of the subject in a case where the next image capturing is to be executed.

3. The medical image diagnosis apparatus according to claim 1,
wherein the processing circuitry is further configured to obtain evaluation information regarding an estimated image estimated to be obtained by image capturing of the diagnosis target region under an image capturing condition different from the first image capturing condition, based on the subject information, and
wherein the evaluation information includes at least either one of first evaluation information regarding image quality of the estimated image and second evaluation information regarding uncertainty of disease classification estimated to be obtained in a case where a disease is classified based on the first medical image and the estimated image.

4. The medical image diagnosis apparatus according to claim 3,
wherein the estimated image is a medical image estimated to be captured in a case where an image of an unimaged portion within the diagnosis target region of the subject is captured, and
wherein the processing circuitry obtains the evaluation information based on the first medical image and the estimated image.

5. The medical image diagnosis apparatus according to claim 3,
wherein the processing circuitry is further configured to generate the learned model by causing a model to execute learning,
wherein the processing circuitry obtains, based on subject information for learning including a medical image for learning, evaluation information for learning regarding an estimated image for learning that is estimated to be obtained by image capturing of a diagnosis target region under an image capturing condition different from an image capturing condition for learning under which the medical image for learning has been captured,
wherein the processing circuitry causes the model to execute learning in such a manner that the evaluation information for learning improves based on the image capturing condition for learning, the subject information for learning, and the evaluation information for learning, and
wherein the processing circuitry determines the second image capturing condition by inputting the subject information and the first image capturing condition to the learned model.

6. The medical image diagnosis apparatus according to claim 1, further comprising:
an X-ray generation unit configured to generate an X-ray; and
an X-ray detector configured to detect the X-ray,
wherein the first image capturing condition and the second image capturing condition include an image capturing position to which the X-ray is emitted, and
wherein an image capturing position included in the second image capturing condition is within an unimaged portion of the diagnosis target region that excludes an image capturing position of the first medical image.

7. The medical image diagnosis apparatus according to claim 1,
wherein the first medical image and the second medical image are magnetic resonance images, and
wherein the first image capturing condition and the second image capturing condition include an image capturing position and a type of an image in magnetic resonance imaging.

8. The medical image diagnosis apparatus according to claim 1, further comprising an ultrasonic probe configured to transmit an ultrasonic wave to the subject,
wherein the first image capturing condition and the second image capturing condition include information regarding a position and an angle of the ultrasonic probe.

9. A medical image processing apparatus, comprising:
processing circuitry configured to:
acquire subject information regarding a subject that includes a first medical image obtained by capturing an image of a diagnosis target region of the subject under a first image capturing condition;
determine a second image capturing condition to capture a second medical image of the subject, based on the subject information and a learned model obtained by executing learning while associating evaluation information regarding a medical image and an image capturing condition; and
output the second image capturing condition to control the medical scanner to perform the scan on the subject by using the medical scanner with the determined second image capturing condition.

10. A method, comprising:
acquiring subject information regarding a subject that includes a first medical image obtained by capturing an image of a diagnosis target region of the subject under a first image capturing condition;
determining a second image capturing condition to capture a second medical image of the subject, based on the subject information and a learned model obtained by executing learning while associating evaluation information regarding a medical image and an image capturing condition;
controlling a medical scanner to perform the scan on the subject by using the medical scanner with the determined second image capturing condition; and
obtaining a medical image of the subject from the medical scanner.
